(19) United States
(12) Patent Application Publication
(10) Pub. No.: US 2007/0190134 A1
(43) Pub. Date: Aug. 16, 2007

(54) Compositions containing nicorandil, preparation method and use

(75) Inventors: Mathieu Nocent, Montrouge (FR); Thierry Bonhomme, Margny les Compiegne (FR)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: Aventis Pharma S.A., Antony (FR)

(21) Appl. No.: 11/642,575

(22) Filed: Dec. 20, 2006

Related U.S. Application Data
(63) Continuation of application No. PCT/FR05/01730, filed on Jul. 5, 2005.

(30) Foreign Application Priority Data
Jul. 8, 2004 (FR) 0407590

(51) Int. Cl. A61K 31/4439; C07D 213/06

(52) U.S. Cl. 424/464; 546/340; 514/343

(57) ABSTRACT

The present invention provides a composition comprising (i) Nicorandil, and (ii) a lubricant selected from a saturated higher aliphatic acid and its salts and/or a saturated higher alcohol, which is solid at ambient temperature, wherein the lubricant is not micronized; processes for preparing such composition; processes for preparing tablets from such composition; and tablets prepared by such processes.

The present invention relates in particular to compositions containing Nicorandil, to the process for preparing them, to tablets containing these compositions, and to their use as a medicament.

More particularly, and according to a first aspect, the invention relates to a composition containing Nicorandil, which has the advantage of allowing the industrial process for the manufacture of tablets containing it to be considerably simplified.

### BACKGROUND

The process currently used at the industrial level for the preparation of Nicorandil (INN) (Ikorel®) tablets involves a granulation step preceding the tablet formation step.

A process comprising a granulation step is described in patent EP 0230932 B1. In said patent, examples 1, 2, 4, 5 and 6 describe processes using a granulation step. In general, it is noted that the use of a granulation step makes it possible to obtain tablets that have a better stability than when this step is absent (tables 1 to 7, examples 3, 7, 8). This is, moreover, one of the reasons for which it was chosen to use a process by granulation for the commercial product.

Commercial excipients usually make it possible to obtain compositions that are acceptable for direct compression. In general, these excipients are in granulated form, and are sold under the name “for direct compression”. Unfortunately, and due to problems of stability inherent in the active ingredient, it has not been possible, up until now, to have a formulation for direct compression that makes it possible to obtain tablets that are sufficiently stable over time. Example 3 of patent EP 0230932 B1 describes a process in which, in a first step, the active ingredient is mixed with stearic acid and then the mixture is micronized. However, the stability of the compositions obtained is not satisfactory (table 3, page 5: 97.3% after 3 months at 40° C., 0% residual water content).

By way of comparison, example 2 (99.4%) is the one which has the stability closest to the commercial composition.

In EP 0230932 B1, page 2, lines 32-36, it is written that an acceptable solution to the problem of tablet stability can be obtained with the mixture of Nicorandil and a saturated aliphatic acid or alcohol. However, this solution is not entirely satisfactory, as can be noted upon reading the stability measurement results, discussed above.

### SUMMARY OF THE INVENTION

Against all expectations, it has been found that it is possible to obtain a composition for direct compression that has a stability equivalent to the best composition obtained via a granulation step, which is the commercial composition.

### BRIEF DESCRIPTION OF THE DRAWINGS

FIG. 1 is a graphic representation of Nicorandil concentration as a function of time for batches stored in blister packs at 25° C., 60% relative humidity.

FIG. 2 is a graphic representation of Nicorandil concentration as a function of time for batches stored in blister packs at 30° C., 65% relative humidity.

FIG. 3 is a graphic representation of Nicorandil concentration as a function of time for batches stored in blister packs at 40° C., 75% relative humidity.

FIG. 4 is a graphic representation of the concentration of impurities as a function of time for batches stored in blister packs at 25° C., 60% relative humidity.

FIG. 5 is a graphic representation of the concentration of impurities as a function of time for batches stored in blister packs at 30° C., 65% relative humidity.

FIG. 6 is a graphic representation of the concentration of impurities as a function of time for batches stored in blister packs at 40° C., 75% relative humidity.

### DETAILED DESCRIPTION OF THE INVENTION

The composition of the invention for direct compression comprises active ingredient (Nicorandil) and a saturated higher aliphatic acid or a saturated higher alcohol that is nonmicronized. An acceptable saturated higher aliphatic acid or saturated higher alcohol must be solid at ambient temperature, i.e. at a temperature close to 20 to 25° C. Preferred saturated higher aliphatic acids or alcohols will also be solid at a temperature in the region of 40° C., preferably of 50° C.

Particularly preferred saturated aliphatic acids can be selected from palmitic acid and stearic acid.

Particularly preferred saturated aliphatic alcohols can be selected from hexadecanoic and octadecanoic alcohols, preferably hexadecan-1-ol and octadecan-1-ol.

A composition according to the invention advantageously comprises (i) Nicorandil, and (ii) a lubricant selected from a saturated higher aliphatic acid and its salts and/or a saturated higher alcohol, which is solid at ambient temperature, in which the lubricant is not micronized.

A preferred lubricant is stearic acid.

A composition according to the invention can also comprise a disintegrating agent and a diluent.

A preferred disintegrating agent is sodium croscarmellose.

A preferred diluent is mannitol.

A composition according to the invention advantageously comprises, by weight, 10% of Nicorandil, and a non-micronized lubricant which is solid at ambient temperature.

A composition according to the invention preferably comprises 8% of nonmicronized stearic acid.

A composition according to the invention advantageously comprises a disintegrating agent, preferably 5% of sodium croscarmellose.

A composition according to the invention advantageously comprises a diluent, preferably mannitol, in particular 76% by weight.

According to a second aspect, the invention relates to a process for preparing a composition according to its first aspect.

In particular, the preparation process according to the second aspect of the invention comprises a first step in which, by weight, 30 parts of Nicorandil, 15 parts of sodium croscarmellose, 35 parts of mannitol and 3 parts of corn starch are mixed so as to form a first pre-mix.

The first pre-mix is preferably calibrated.

The process according to the invention can also comprise a second step in which the calibrated first pre-mix is mixed with 193 parts by weight of mannitol so as to form a second pre-mix.

The process according to the invention can also comprise a third step in which the second pre-mix is mixed with 24 parts by weight of nonmicronized stearic acid.

According to a third aspect, the invention relates to a composition for direct compression, obtained by means of a process according to its second aspect.

According to a fourth aspect, the invention relates to a process for preparing a tablet comprising Nicorandil, which comprises a first step (i) in which a composition for direct compression according to its third aspect is placed in an impression of a mold, which comprises a second step (ii) in which a counter-impression of the mold is applied against the impression in such a way that the composition for direct compression is trapped in a chamber of volume V1 of the mold, and which also comprises a third step (iii) in which the volume V1 of the mold is reduced to a volume V0 less than the volume V1 by compression until a tablet is obtained.

The process according to its fourth aspect advantageously comprises a fourth step (iv), in which the impression and the counter-impression are separated and the tablet is extracted from the chamber.

According to a fifth aspect, the invention relates to a tablet obtained according to its fourth aspect.

According to a sixth aspect, the invention relates to an acceptable packaging for tablets according to the fifth aspect, in particular a blister pack or a bottle.

The advantages of the invention will be more particularly illustrated by the following example:

An acceptable composition comprising Nicorandil according to the prior art can be prepared as follows:

1) Commercial Composition:

TABLE 1

| | Centesimal | |
|---|---|---|
| | theoretical formula | Operating unit |
| Constituent name | (%) | (kg) |
| Nicorandil | 10.00 | 24.10 |
| Sodium | 5.00 | 12.00 |
| croscarmellose | | |
| Mannitol 200 SD | 76.00 | 183.14 |
| Corn starch | 1.00 | 2.40 |
| Micronized | 8.00 | 19.46 |
| stearic acid | | |
| Operating unit | 100 | 241.10 i.e. |
| | | 2 411 000 |
| | | tablets of Ikorel |
| | | 10 mg or |
| | | 1 205 500 tablets |
| | | of Ikorel 20 mg |

2) Preparation Process of the Prior Art (Commercial Process):

Phase 1: Preparation of the Ikorel Neutral Granule (Table 2, Below)

TABLE 2

| Materials | Amounts | | Procedures |
|---|---|---|---|
| Simple mannitol | 430.379 | kg | WEIGHING OUT OF |
| Corn starch | 5.640 | kg | STARTING |
| Stearic acid | 33.981 | kg | MATERIALS |
| Mannitol + stearic acid | 464.360 | kg | MIXING and |
| | | | PREHEATING |
| Cold purified water | 7.527 | kg | PREPARATION OF |
| Boiling purified water | 82.735 | kg | THE WETTING |
| Corn starch | 5.640 | kg | SOLUTION |
| N.A | 470 | kg | GRANULATION |
| N.A | 470 | kg | DRYING AND |
| | | | COOLING |

| | | |
|---|---|---|
| N.A | approximately | CALIBRATION |
| | 470 kg | |
| N.A | 400 kg | LOADING INTO |
| | (elimination of | CONTAINER |
| | surplus) | |

Phase 2: Manufacture of Ikorel Tablets

TABLE 3

| Materials | Amounts | | Procedures |
|---|---|---|---|
| Nicorandil | 24.10 | kg | WEIGHING OUT OF |
| Stearic acid | 5.00 | kg | STARTING |
| Sodium croscarmellose | 12.00 | kg | MATERIALS |
| Nicorandil | 24.10 | kg | PREMIXING |
| Ikorel neutral granule | 80.00 | kg | |
| Nicorandil/neutral | 104.10 | kg | CALIBRATION |
| granule pre-mix | | | |
| Stearic acid | 5.00 | kg | |
| Sodium croscarmellose | 12.00 | kg | |
| Calibrated pre-mix | 121.10 | kg | FINAL MIXING |
| Ikorel neutral granule | 120.00 | kg | |
| Final mix | 241.10 | kg | COOLING OF THE |
| | | | FINAL MIX |
| Final mix | 241.10 | kg | COMPRESSION |

An acceptable composition comprising Nicorandil according to the invention can be prepared as follows:

1) Composition According to the Invention:

TABLE 4

| | Centesimal | |
|---|---|---|
| | theoretical | Operating unit |
| Constituent name | formula (%) | (kg) |
| Nicorandil | 10.00 | 30.00 |
| Sodium croscarmellose | 5.00 | 15.00 |
| Mannitol 200 SD | 76.00 | 228.00 |
| Corn starch | 1.00 | 3.00 |
| Nonmicronized stearic | 8.00 | 24.00 |
| acid | | |
| OPERATING UNIT | 100 | 300.00 i.e. |
| | | 3 000 000 |
| | | Ikorel 10 mg |
| | | tablets or |
| | | 1 500 000 Ikorel |
| | | 20 mg tablets |

2) Preparation Process According to the Invention:

TABLE 5

| MATERIALS | Amounts | | Procedures |
|---|---|---|---|
| Nicorandil | 30.00 | kg | WEIGHINGS OUT |
| Sodium croscarmellose | 15.00 | kg | |
| Mannitol 200 SD | 193.00 | kg | |
| Mannitol 200 SD | 35.00 | kg | |
| Corn starch | 3.00 | kg | |
| Nonmicronized stearic | 24.00 | kg | |
| acid | | | |
| Nicorandil | 30.00 | kg | PRE-MIXING 1 |
| Sodium croscarmellose | 15.00 | kg | |
| Mannitol 200 SD | 35.00 | kg | |
| Corn starch | 3.00 | kg | |
| Pre-mix 1 | 83.00 | kg | CALIBRATION |
| Calibrated pre-mix 1 + | 83.00 | kg | PRE-MIXING 2 |
| Mannitol 200 SD | 193.00 | kg | |
| Pre-mix 2 + | 276.00 | kg | FINAL MIXING |
| Nonmicronized stearic | 24.00 | kg | |
| acid | | | |
| Final mix | 300.00 | kg | COOLING OF THE |
| | | | FINAL MIX |
| Final mix | 300.00 | kg | COMPRESSION |

Comparison Between the Stability of the Commercial Composition and of the Composition According to the Invention when the Compositions are in the Form of Loose Tablets, Stored in a Minibag:

1) Commercial Composition:

TABLE 6

| | Water | | | Dissolution |
|---|---|---|---|---|
| Sampling | content | Nicorandil | Impurities | value |
| date | (%) | (mg/tablet) | (μg/tablet) | (UV, as %) |
| t = 0 | 0.2 | 9.7 | 12 | 98 |
| 1 month | 0.2 | 9.7 | 47 | 102 |
| 2 months | 0.4 | 9.8 | 74 | 104 |
| 3 months | 0.3 | 9.7 | 102 | 102 |
| 4 months | 0.3 | 9.5 | 141 | 101 |
| 5 months | 0.3 | 9.7 | 175 | 101 |

2) Composition According to the Invention:

TABLE 7

| | Water | | | Dissolution |
|---|---|---|---|---|
| Sampling | content | Nicorandil | Impurities | value |
| date | (%) | (mg/tablet) | (μg/tablet) | (UV, as %) |
| t = 0 | 0.3 | 10.0 | 43 | 108 |
| 1 month | 0.1 | 9.9 | 52 | 103 |
| 2 months | 0.2 | 9.8 | 75 | 103 |
| 3 months | 0.1 | 9.6 | 101 | 103 |
| 4 months | 0.1 | 9.7 | 101 | 99 |
| 5 months | 0.1 | 9.7 | 124 | 98 |

The loose tablets obtained by means of the process according to the invention are more stable than the commercial tablets.

The water contents, an important factor in stability, are systematically lower for the batch of product obtained via the process according to the invention.

The amounts of Nicorandil are as stable over time for the tablets according to the invention as for the commercial tablets.

Impurity values that are higher at t=0 for the tablets according to the invention compared with the commercial tablets are noted. However, the increase in the level of impurities is slower over time for the tablets according to the invention. Thus, at five months, impurity values that are outside the standards are obtained for the commercial batch, which is not the case of the batch according to the invention.

Finally, the dissolution values are stable in the two cases.

Compared Stability of the Tablets According to the Storage Conditions, at 6 Months, 40° C., 75% RH, in Blister Packs:

TABLE 14

| | Nicorandil | Impurities |
|---|---|---|
| | (mg/tablet) | (μg/tablet) |
| Batch 20 CMP | | |
| T0 | 9.9 | 30 |
| T 3 months | 8.3 | 1499 |
| T 6 months | 7.2 | 2670 |
| Batch 21 CMP | | |
| T0 | 10.0 | 33 |
| T 3 months | 7.7 | 2492 |
| T 6 months | 5.0 | 2665 |
| Batch 22 CMP | | |
| T0 | 9.9 | 24 |
| T 3 months | 7.9 | 1834 |
| T 6 months | 6.9 | 2336 |
| Batch LOP107 CD | | |
| T0 | 10.0 | 43 |
| T 3 months | 8.0 | 1548 |
| T 6 months | 7.1 | 2319 |

The batches 20, 21 and 22 CMP are obtained by means of the current commercial process. The batch LOP107 CD is a batch obtained by means of the process according to the invention, described above.

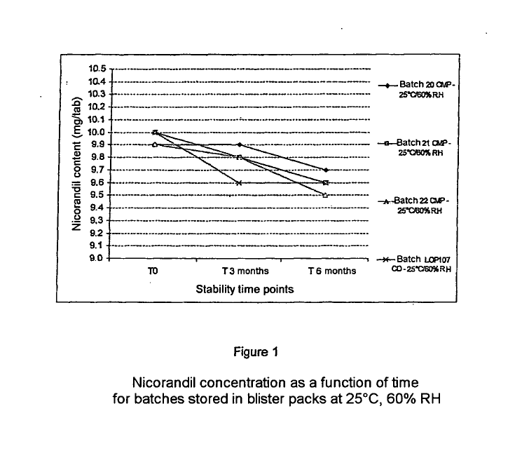

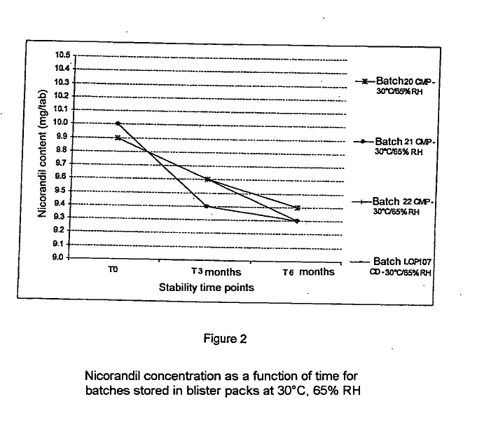

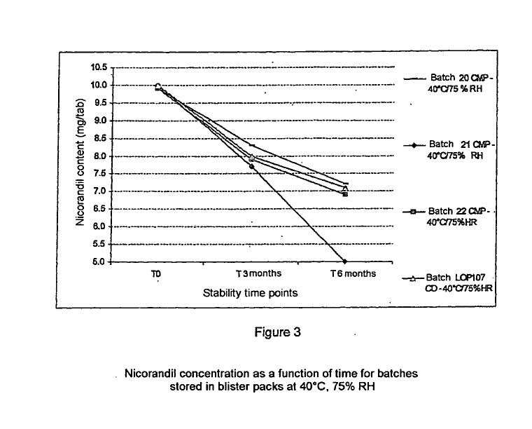

FIGS. 1 to 3 are graphic representations of the evolution of Nicorandil content as a function of time for, respectively, tablets stored in blister packs at 25° C., 60% RH; 30° C., 65% RH; and 40° C., 75% RH; the latter corresponding to the values presented in table 14.

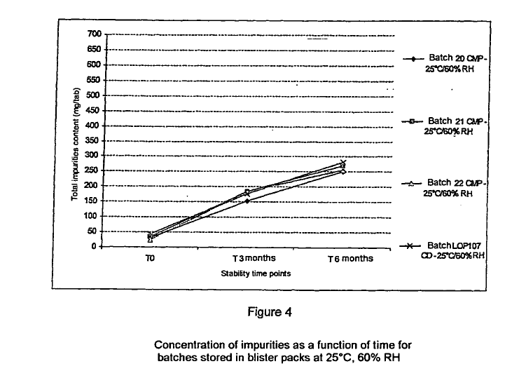

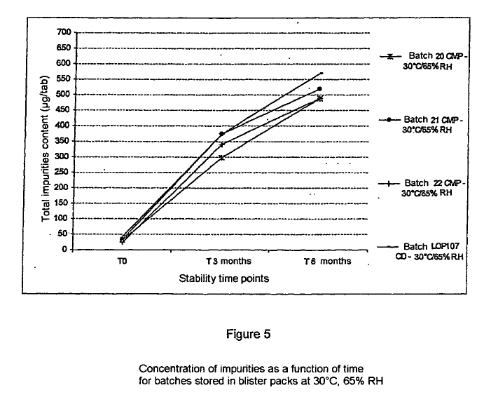

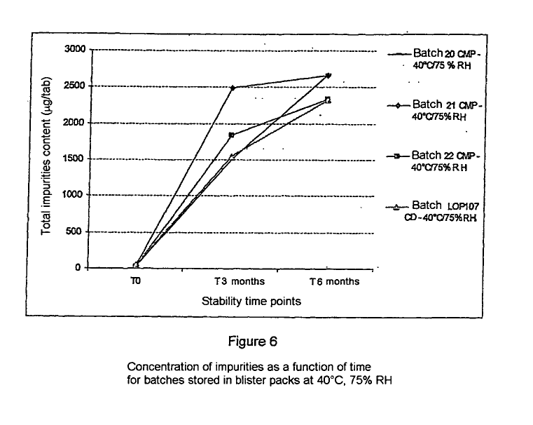

FIGS. 4 to 6 are graphic representations of the evolution of impurity content as a function of time for, respectively, tablets stored in blister packs at 25° C., 60% RH; 30° C., 65% RH; and 40° C., 75% RH; the latter corresponding to the values presented in table 14.

Whether at 25° C., 60% RH or at 30° C., 65% RH, at t=6 months, the content of active ingredient of the batch obtained by means of the process according to the invention is equivalent to that measured in the batches obtained by means of the current process. The same is true of the impurity concentrations.

At 40° C., 75% RH, at t=6 months, the content of active ingredient of the batch obtained by means of the process according to the invention is better than or equivalent to that measured in the batches obtained by means of the current process. The same is true of the impurity concentrations.

The loose tablets, manufactured according to the process by direct compression, and stored in a minibag, are more stable than the commercial tablets.

From this additional study, it appears that the tablets according to the invention exhibit stability qualities that are entirely noteworthy with respect to the tablets according to the current process. It is necessary to recall that the stability conditions considered here are drastic conditions for this product, for which strict storage conditions are recommended (temperature<25° C.).

What is claimed is:

1. A composition comprising (i) Nicorandil, and (ii) a lubricant selected from a saturated higher aliphatic acid and its salts and/or a saturated higher alcohol, which is solid at ambient temperature, wherein the lubricant is not micronized.

2. A composition according to claim 1 comprising (i) Nicorandil, and (ii) a lubricant wherein the lubricant is a saturated higher aliphatic acid, or a salt of said acid, said lubricant being solid at ambient temperature, wherein the lubricant is not micronized.

3. A composition according to claim 1 comprising (i) Nicorandil, and (ii) a lubricant wherein said lubricant is a saturated higher alcohol, said lubricant being solid at ambient temperature, wherein the lubricant is not micronized.

4. A composition according to claim 1, wherein the lubricant is stearic acid.

5. A composition according to claim 2, wherein the lubricant is stearic acid.

6. A composition according to claim 1, further comprising a disintegrating agent and a diluent.

7. A composition according to claim 6, wherein the disintegrating agent is sodium croscarmellose.

8. A composition according to claim 6, wherein the diluent is mannitol.

9. A composition comprising, by weight, 10% of Nicorandil, and a nonmicronized lubricant which is solid at ambient temperature.

10. A composition according to claim 9, comprising 8% of nonmicronized stearic acid.

11. A composition according to claim 9, further comprising a disintegrating agent.

12. A compositions according to claim 11, comprising 5% of sodium croscarmellose.

13. A composition according to claim 9, further comprising a diluent.

14. A composition according to claim 13, comprising 76% of mannitol.

15. A process for preparing a composition according to claim 1, comprising a first step in which, by weight, 30 parts of Nicorandil, 15 parts of sodium croscarmellose, 35 parts of mannitol and 3 parts of corn starch are mixed so as to form a first pre-mix.

16. A process according to claim 15, wherein the first pre-mix is calibrated.

17. A process according to claim 16, comprising a second step in which the calibrated first pre-mix is mixed with 193 parts by weight of mannitol so as to form a second pre-mix.

18. A process according to claim 17, comprising a third step in which the second pre-mix is mixed with 24 parts by weight of nonmicronized stearic acid.

19. A composition for direct compression, obtained by means of a process according to claim 15.

20. A composition for direct compression, obtained by means of a process according to claim 18.

21. A process for preparing a tablet comprising Nicorandil, comprising a first step (i) in which a composition for direct compression as claimed in claim 16 is placed, at ambient temperature, in an impression of a mold; a second step (ii) in which a counter-impression of the mold is applied against the impression in such a way that the composition for direct compression is trapped in a chamber of volume V1 of the mold; and a third step (iii) in which the volume V1 of the mold is reduced to a volume V0 less than the volume V1 by compression until a tablet is obtained.

22. A process according to claim 21, further comprising a fourth step (iv), in which the impression and the counter-impression are separated and the tablet is extracted from the chamber.

23. A tablet obtained according to claim 22.
